(19) United States
(12) Patent Application Publication
(10) Pub. No.: US 2006/0115905 A1
(43) Pub. Date: Jun. 1, 2006

(54) Microscale diffusion immunoassay in hydrogels

(75) Inventors: Anson Hatch, Mountlake Terrace, WA (US); Paul Yager, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: University of Washington, Seattle, WA (US)

(21) Appl. No.: 11/165,619

(22) Filed: Jun. 23, 2005

Related U.S. Application Data
(63) Continuation of application No. 10/277,047, filed on Oct. 21, 2002, now abandoned.
Application No. 10/277,047 is a continuation-in-part of application No. 09/574,797, filed on May 19, 2000, now Pat. No. 6,541,213.
Application No. 09/574,797 is a continuation-in-part of application No. 09/503,563, filed on Feb. 14, 2000, now abandoned.
Application No. 09/574,797 is a continuation-in-part of application No. 09/426,683, filed on Oct. 25, 1999, now abandoned.
Application No. 09/426,683 is a continuation of application No. 08/829,679, filed on Mar. 31, 1997, now Pat. No. 5,972,710.
Application No. 08/829,679 is a continuation-in-part of application No. 08/625,808, filed on Mar. 29, 1996, now Pat. No. 5,716,852.
(60) Provisional application No. 60/135,417, filed on May 21, 1999.
(60) Provisional application No. 60/346,054, filed on Oct. 19, 2001.

(51) Int. Cl. G01N 33/557; G06K 9/00

(52) U.S. Cl. 436/517; 382/128

(57) ABSTRACT

A diffusion immunoassay (DIA) for determining the presence and concentration of analyte particles by detecting the diffusion front. A hydrogel containing immobilized binding particles is placed in contact with a carrier fluid containing analyte particles, which analyte particles diffuse into the hydrogel and bind with the immobilized binding particles. A detection device detects the position of the diffusion front formed in the hydrogel to determine the presence and concentration of the analyte particles which have diffused into the hydrogel.

### CROSS-REFERENCE TO RELATED APPLICATIONS

This application claims priority from U.S. Provisional Application No. 60/346,054, filed Oct. 19, 2001. This application also claims priority from U.S. application Ser. No. 09/574,797 filed May 19, 2000, which application is a continuation-in-part of U.S. application Ser. No. 09/503,563 filed Feb. 14, 2000, now abandoned, which claims priority from U.S. Provisional Application No. 60/135,417 filed May 21, 1999. This application also claims priority from U.S. application Ser. No. 09/426,683 filed Oct. 25, 1999, which is a continuation of U.S. application Ser. No. 08/829,679 filed Mar. 31, 1997, now U.S. Pat. No. 5,972,710, which is a continuation-in-part of U.S. application Ser. No. 08/625,808 filed Mar. 29, 1996, now U.S. Pat. No. 5,716,852.

### BACKGROUND OF THE INVENTION

1. Field of the Invention

This invention relates generally to microscale devices for performing analytical testing and, in particular, to a microscale diffusion immunoassay (DIA) for determining presence and concentration of analytes by exploiting molecular binding reactions and differential diffusion rates using hydrogels.

2. Description of the Prior Art

The immunoassay is the workhorse of analytical biochemistry. It allows the unique binding abilities of antibodies to be widely used in selective and sensitive measurement of small and large molecular analytes in complex samples. The driving force behind developing new immunological assays is the constant need for simpler, more rapid, and less expensive ways to analyze the components of complex sample mixtures. Current uses of immunoassays include therapeutic drug monitoring, screening for disease or infection with molecular markers, screening for toxic substances and illicit drugs, and monitoring for environmental contaminants.

Flow injection immunoassays have taken advantage of specific flow conditions (U. de Alwis and G. S. Wilson, Anal. Chem. 59, 2786-9 (1987)), but also use high Reynolds number effects for mixing. Micro-fabricated capillary electrophoresis devices, which are truly microfluidic, have been used for rapidly separating very small volumes of immunoreagents following binding reactions (N. Chiem and D. J. Harrison, Anal. Chem. 69, 373-8 (1997)). One of the unique features of microfluidic devices that has yet to be exploited for immunoassay development is the presence of laminar flow under low Reynolds number conditions. Laminar flow allows quantitative diffusional transport between adjacent flowing streams, while retaining the relative positions of non-diffusing components such as cells and larger microspheres. While these conditions are impediments to application of some macro-scale techniques, they allow creation of new types of analyses that are uniquely well suited to microfluidic systems, such as the H-Filter for extraction of solutes (J. P. Brody, P. Yager, R. E. Goldstein, R. H. Austin, Biophysical Journal 71(6), 3430-3441(1996); U.S. Pat. No. 5,932,100; J. P. Brody and P. Yager, Sensors and Actuators A (Physical) A58(1), 13-18 (1997); the V-Groove device for low-volume flow cytometry; U.S. Pat. No. 5,726,751, the T-Sensor for detection of diffusable analytes (A. E. Kamholz, B. H. Weigl, B. A. Finlayson, P. Yager, [1999] Anal. Chem., 71(23):5340-5347; U.S. Pat. No. 5,716,852; U.S. Pat. No. 5,972,710; B. H. Weigl and P. Yager, Science 283, 346-347 [1999]; R. B. Darling, J. Kriebel, K. J. Mayes, B. H. Weigl, P. Yager, Integration of microelectrodes with etched microchannels for in-stream electrochemical analysis, .mu.TAS '98, Banff, Canada [1998]; B. H. Weigl and P. Yager, Sensors and Actuators B (Chemical) B39 (1-3), 452-457 [1996]; B. H. Weigl, M. A. Holl, D. Schutte, J. P. Brody, P. Yager, Anal. Methods & Instr., 174-184 [1996]; B. H. Weigl, et al., Simultaneous self-referencing analyte determination in complex sample solutions using microfabricated flow structures (T-Sensors), .mu.TAS '98, Banff, Canada [1998]) and others as described in U.S. Pat. Nos. 5,922,210; 5,747,349; 5,748,827; 5,726,404; 5,971,158; 5,974,867 and 5,948,684; WO 98/43066 published Oct. 1, 1998; U.S. Ser. No. 08/938,584 filed Sep. 26, 1997; WO 99/17100 published Apr. 8, 1999; WO 99/17119 published Apr. 8, 1999; U.S. Ser. No. 09/196,473 filed Nov. 19, 1998; U.S. Ser. No. 09/169,533 filed Oct. 9, 1998; WO 99/60397 published Nov. 25, 1999; U.S. Ser. No. 09/404,454 filed Sep. 22, 1999; and Ser. No. 09/464,379, filed Dec. 15, 1999 for “Magnetically-Actuated Fluid Handling Devices for Microfluidic Applications.”

All publications referred to herein are hereby incorporated by reference in their entirety to the extent not inconsistent herewith.

U.S. patent application Ser. No. 09/574,797, which application is hereby incorporated by reference, teaches a method for detecting the presence of analyte particles comprising providing binding particles capable of binding with said analyte particles; providing a system in which at least one of said binding particles and said analyte particles can diffuse toward the other; providing means for detecting any of said particles or complexes between them, or a diffusion front of said binding particles, said analyte particles, or said complexes in said system, and detecting said particles or complexes or said diffusion front. When said analyte particles and said binding particles meet and bind to each other, a slowing of the particles or a diffusion front may be detected as an indication of the presence of said analyte particles. The binding particles, or the analyte particles, or complexes between them must be visible or detectable, e.g. by optical or electrical detection means or other detection means known to the art, or must be labeled to become visible or detectable.

The '797 application also provides a device for determining the presence or concentration of sample analyte particles in a medium comprising: means for contacting a first medium containing analyte particles with a second medium containing binding particles capable of binding to said analyte particles; wherein at least one of said analyte or binding particles is capable of diffusing into the medium containing the other of said analyte or binding particles; and means for detecting the presence of diffused particles. One or both of the analyte and binding particles may be labeled or unlabeled.

Systems allowing diffusion of analyte or binding particles toward each other can be systems in which fluids containing analyte particles (referred to herein as analyte fluids) are placed in contact with fluids containing binding particles (referred to herein as “diffusion fluids”), or fluids containing analyte particles, are placed in contact with solids containing binding particles capable of diffusing into the analyte fluid. Or, the system may be one in which fluids containing binding particles are placed in contact with solids containing analyte particles capable of diffusing into the diffusion fluids. Such systems can be flowing or stationary systems, or can comprise fluids separated by membranes capable of allowing diffusion of analyte and/or binding particles therethrough, or can comprise two fluids containing analyte and binding particles respectively separated by a removable barrier, which is removed to allow diffusion to take place.

The flowing systems which are described in the '797 application give rise to stationary diffusion profiles. The position of such stationary diffusion profiles are used to determine concentration of analyte particles. Often, the analyte and diffusion streams must flow in contact for a significant period of time to form a stable diffusion profile at the detection area. This leads to larger devices and increased reagent volumes.

The diffusion immunoassay taught in the '797 application relies on interfacing two solutions and monitoring the diffusion of components across the interface. Using laminar flow to interface solutions requires precise and sustained fluid delivery. An attractive alternative is to use the structural stability of a hydrogel to interface two solutions rather than laminar flow. The aim of this invention is to develop a diffusion analysis using acrylamide hydrogels to interface a solution with the hydrogel solvent. This offers several advantages over a laminar flow system including simplified fluid delivery, conservation of reagent volumes and device space, and reducing confounding effects of hydrodynamic flow. Additionally, the porous nature of a hydrogel can be tuned to discriminate between molecules of different size and other properties such as charge by changing the monomeric components. This would serve to enhance differences in diffusivity between molecules for more effective diffusion based separation and analysis.

### SUMMARY OF THE INVENTION

It is therefore an object of the present invention to provide a diffusion binding assay in which continuous flow is not necessary.

It is a further object of the present invention to provide a diffusion binding assay which greatly reduces device area and reagent volumes.

It is a still further object of the present invention to provide a diffusion binding assay which greatly simplifies fluid handling.

These and other objects of the present invention will be more readily apparent in the description and drawings which follow.

### BRIEF DESCRIPTION OF THE DRAWINGS

FIG. 1 is a schematic representation of the diffusion immunoassay of the present invention;

FIGS. 2 A-D show the present invention with and without binding molecules at different times;

FIG. 3 is a graph showing diffusion profiles of fluorescent biotin imaged at a distance from the contact junction shown in FIG. 1 for several samples;

FIG. 4 is a schematic representation of the present invention showing the positioning of the detection means; and

FIG. 5 is a chart showing the apparent diffusivity of molecules in different gel concentrations.

### DETAILED DESCRIPTION OF THE PREFERRED EMBODIMENTS

Microfluidics is rapidly becoming a cornerstone technology as chemical diagnostics and the microfluidics diffusion immunoassay (DIA) or diffusion binding assay (DBA) of the present invention is a useful tool for many diagnostic applications.

By taking advantage of differences in the diffusion coefficients of small molecules bound and unbound to much larger molecules (either larger molecules free in solution or molecules smaller to larger in size that are immobilized), this invention provides a binding assay format offering many advantages over conventional formats. This diffusion binding assay (DBA) is well suited to implementation using microfluidic technology, which offers the advantages of small reagent and sample volumes, continuous monitoring capabilities, low-cost mass production of devices, and integrated testing networks amenable to automation.

Analyte particles may be molecules, preferably having a molecular weight range between about 100 and about 1,000,000, or particles of corresponding size. The term “sample antigen”, as used herein, refers to analyte particles. Analyte particles may also be antibodies.

Analyte particles which may be used in DIA systems include, but are not limited to, abused drugs such as amphetamine and methamphetamine, barbiturates, benzodiazepines, bensodiazepine in serum, cannabinoids, cocaine metabolites, ethanol, methadone, opiates, phencyclidine, propoxyphene, salicylate, tricyclic and antidepressants; cancer drugs such as methotrexate; fertility and pregnancy drugs such as free estriol, selected prolactins, and total estriol; medications for heart disease; anti-inflammatories; drugs which require therapeutic monitoring such as amikacin, carbamazepine, digitoxin, digoxin, disopyramide, ethosuximide, free carbamazepine, free phenyloin, free valproic acid, gentamicin, lidocaine, N-acetylprocainamide, netilmicin, Phenobarbital, phenyloin, primidone, procainamide, quinidine, theophylline, tobramycin, valproic acid, vancomycin; endogenous molecules such as thyroid; antigens detected in assay systems such at T-Uptake, including T4; antigens used in transplant monitoring including assays of cyclosporine, serum cyclosporine, cyclosporine in whole blood, and coritsol. Potential drug molecules that bind to either serum proteins such as albumin or alpha-1-acid glycoprotein, enzymes, or antibodies. Proteins that bind specifically to DNA fragments such as promoter sequences or vice versa. DNA or RNA fragments that hybridize to matching DNA or RNA.

The analyte fluid may be an aqueous solution containing the antigen, a bodily fluid such as whole blood, serum, saliva, urine or other fluid, contaminated drinking water, fermentation broths, samples from industrial processes requiring monitoring, extracted fluid from cells, samples containing molecules from a chemical library, solutions containing PCR amplified DNA fragments, or any other fluid for which analysis is required.

Detectable markers or labeling agents for labeling the analyte particles or binding particles include any particles capable of binding or adhering to the analyte particles and not interfering with binding of the binding particle selected for the assay. Labeling agents may include fluorescent, phosphorescent, chemiluminescent, enzyme particles, and other labeling agents known to the art. The term “labeled antigen” as used herein refers to labeled analyte particles. Labeling agents should be small enough to provide label/analyte particle complexes which are smaller in size than the binding molecule or binding molecule and immobilizing counterpart so that diffusion coefficients of the labeled analyte particles are larger than the diffusion coefficient of the complex formed upon binding to the binding molecule. For example, an analyte particle having a molecular weight of 10,000 might be labeled with a molecule having a molecular weight of about 100 to 100,000 as long as the binding molecule was of molecular weight 200,000 or larger or was of molecular weight of 100 or larger and immobilized by a solid or particle of molecular weight 200,000 or larger. The label may be soluble or insoluble in the fluid and may adhere to the analyte particle by adsorption, absorption or chemical binding. For example, the labeling agent can be a conventional art-known dye, a metal particle, or any other detectable particle known to the art.

The term “particles” includes molecules, cells, large molecules such as proteins, small molecules comprised of one or several atoms, and ions. The particles may be suspended or dissolved in the carrier fluid. The term “particles” as used herein does not include the molecules of the carrier fluid.

The binding particle may be any particle capable of binding or adhering, e.g., by covalent or ionic binding, absorption or other means known to the art, to the analyte particle and with the labeled analyte particle to form complexes with a diffusion coefficient greater than that of the analyte particle and labeled analyte particle. Preferably the diffusion coefficient of the complex is very much greater than that of the labeled analyte particles, and should be at least about two to five times greater than that of the labeled analyte particles, more preferably at least about ten times greater than that of the labeled analyte particles. Preferably the binding particle is at least as large as the analyte particle or the binding particle is immobilized by a solid or particle larger than the analyte particle. The binding particle may be a protein, enzyme, DNA fragment, antibody, either monoclonal or polyclonal, or a synthetic binding particle made using a combinatorial process to provide a specific binding site, or a particle of a substance such as activated charcoal capable of adhering to the labeled analyte particle. Binding particles as defined above may also function as analyte particles, e.g. antibodies may function as analyte particles herein.

The “diffusion front” (also referred to as “diffusion profile” herein) is a detectable edge or line created by diffusing particles. It may be more or less sharp or diffuse depending on system parameters such as relative amounts of analyte and binding particles, relative diffusion coefficients of both, amount of labeling, viscosities of the system, and other parameters known to the art. The term “slowing” with reference to the diffusion front includes stopping, as well as any detectable amount of slowing. The “diffusion front” may include a detectably more intense area or line closer to the point(s) from which diffusion of particles begins caused by complexing of labeled particles to form slower-diffusing complexes, with relatively less intense areas further from said points caused by uncomplexed labeled particles; or the “diffusion front” may be the absolute border of the area into which particles have diffused.

Slowing of the diffusion front may be observed or detected; or the position of the diffusion front after a predetermined time from when the particles begin diffusing may be observed or otherwise detected and compared with a similar calibration or control system or systems containing known amounts of analyte particles, e.g. from 0 to any typical concentration. In this way, concentration as well as presence of analyte particles can be determined.

The devices of this invention may comprise detecting means external to the device for detecting the diffusion profile. Detection and analysis is done by any means known to the art, including optical means, such as optical spectroscopy, light scattering, and other means such as absorption spectroscopy or fluorescence, electrical means, e.g., electrodes inserted into the device, or virtually any microanalytical technique known to the art including magnetic resonance techniques, or other means known to the art to detect the diffusion profile. Preferably optical, fluorescent or chemiluminescent means are used. More preferably the labels used for the analyte particles are fluorescent and detection is done by means of a CCD camera or a scanning laser with a photomultiplier. In the latter, a laser is scanned back and forth across the device by means of a piezoelectric drive. A photo multiplier tube is placed to detect the position of the laser spot and coupled to software to calculate the diffusion profile from the laser signal and position.

Computer processor means may be used to determine the presence or concentration of the analyte particles from the detected diffusion profile. The processor may be programmed to compare the diffusion profile with diffusion profiles taken using varying known concentrations of analyte, e.g., calibration curves or diffusion profiles in reference streams.

The present invention is a system in which it is not necessary that the substances containing the analyte particles and the binding particles be in parallel laminar flow. All that is required that they be in contact for a sufficient period of time to form a diffusion profile indicative of the concentration of analyte particles.

A hydrogel platform may offer a convenient means of simplifying the fluid delivery system for diffusion based analysis. In the simplest case, a cartridge containing multiple hydrogel wells could be loaded with sample by capillary action. A number of theoretical and experimental studies of molecular diffusion in hydrogels are relevant to the development of a hydrogel platform for diffusion based binding analysis. Molecular diffusion in hydrogels is of general interest for understanding physiological systems such as molecular transport through membranes and tissue, for designing gels as a separation media, and for designing gels for the controlled release of drugs. A large number of polymers and composites have been studied and characterized for these purposes. Acrylamide has been shown to produce the desired effect on the permeability of molecules of interest, and is neutrally charged and proven to be relatively inert as a separation media. Gels formed with acrylamide usually have very uniform properties and can be formed with a range of pore sizes depending on initial concentrations of monomer and cross-linker and on the polymerization conditions.

A hydrogel serves as a porous mechanical structure that acts as a barrier to hydrodynamic flow while allowing at least some portion of the solute to enter by diffusion. This can be especially advantageous when dealing with samples that have variable viscosity or viscosity different from that of the buffer. This also simplifies fluid delivery options allowing an assay to be conducted without precisely controlled pumping and eliminating much of the plumbing that occupies valuable microdevice real estate. For diffusion type assays that require long times, much less device space and fluid is necessary for a hydrogel device than the T-Sensor.

The device of this invention can detect analytes present in a carrier liquid at concentrations less than about 1 μM, preferably less than about 100 nM, and more preferably less than about 2.5 nM.

Binding particles, preferably antibodies or proteins, may be present at any concentration providing visible results. Preferably at least about a five to ten-fold excess concentration of binding particles in the gel is used based on the estimated amount of analyte particles. As will be appreciated by those skilled in the art, higher concentrations of binding particles are also useful.

Preferably the hydrogel is formed to incorporate the binding particles, e.g., by polymerization in the presence of binding particles. Polymerization may be conducted by means known to the art, e.g. photopolymerization or chemically initiated polymerization. The hydrogel is preferably solid enough not to flow into the carrier for the analyte particles, but not so solid as to prevent diffusion of analyte particles into it within the time periods contemplated for the assays.

This invention also provides devices such as containers comprising antibody-loaded hydrogels with space for adding carriers comprising analyte particles, or containers containing multiple antibody-loaded hydrogels, loaded with the same or different antibodies, to which carrier comprising analyte particles may be added.

These devices may be incorporated into computer-controlled processing systems for performing multiple assays on carriers for the same analyte, or different analytes, or on different carriers containing the same or different analytes.

The diffusion coefficient of the analyte is preferably larger than that of the binding particle, preferably in the range of about ten times that of the binding particle; however, so long as the analyte diffuses into the hydrogel in measurable amounts while the binding particle is not lost by diffusion into the carrier for the analyte particle in amounts sufficient to interfere with measurement of the analyte concentration, any size analyte or binding particle may be used. The binding particle may be immobilized on larger particles such as plastic beads if desired to reduce diffusion of the binding particles.

A gel concentration of about 5% to about 10%, preferably about 7.5%, may be used, with a ratio of acrylamide monomer crosslinker of at least about 30:1 to about 45:1, preferably about a 37.5:1 ratio of acrylamide monomer: crosslinker in this invention.

The method is preferably performed in less than about five minutes, preferably less than or equal to about three minutes, and more preferably less than or equal to about one minute.

A preferred carrier is blood or a blood product such as plasma. Analytes in other biological fluids may also be detected by the methods, apparatuses and systems of this invention. Analytes in any and all fluids (including gas and liquids) may be detected by these methods, apparatuses and systems. One advantage to using blood in this invention is that blood cells do not diffuse into the hydrogel, eliminating the need for centrifugation to remove cells.

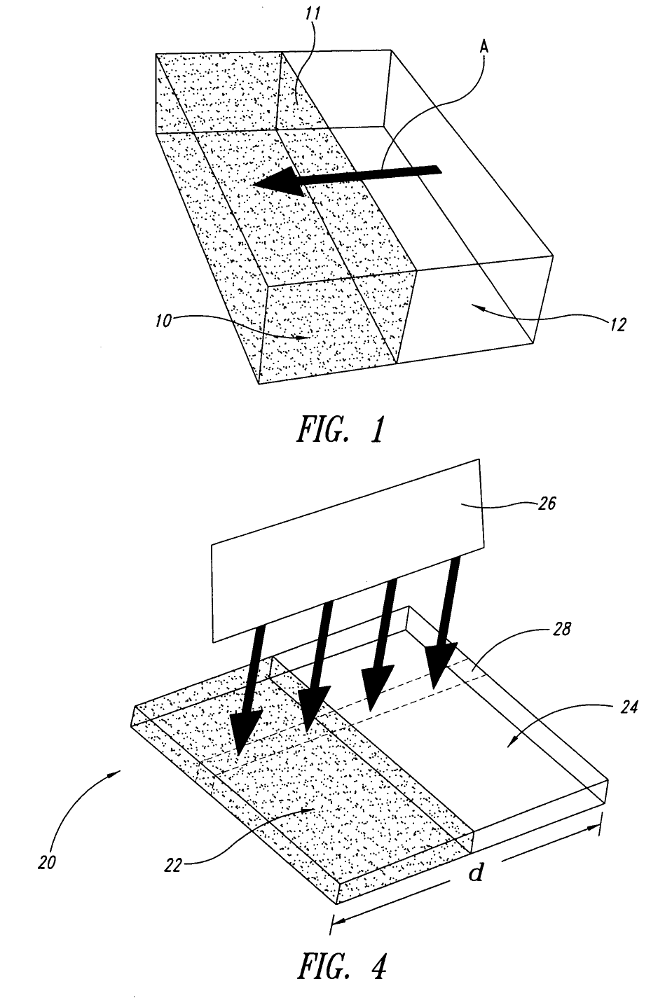

FIG. 1 is a graphic representation of a diffusion immunoassay (DIA) using a hydrogel. Referring now to FIG. 1, there is shown a precast hydrogel 10 containing an immobilized antibody. A sample 12 containing the analyte of interest is spiked with labeled analyte and placed in contact with hydrogel 10, which contains the analyte-specific antibody. The analyte particles can be fluorescently labeled, or labeled by other detectable means known in the art, so that the position of the particles diffused into hydrogel 10 and/or bound to the binding particles can be detected. Other means of detecting diffused antigen particles known in the art may also be used, such as those described in U.S. Pat. Nos. 6,297,061 and 6,221,677, along with application Ser. No. 09/804,780, which are incorporated herein by reference to the extent not inconsistent herewith. The labeled and unlabeled analytes are then allowed to diffuse for a short time (seconds to minutes) into hydrogel 10 in the direction of arrow A across the hydrogel-sample contact plane 11, where they compete for the available antibody binding sites. The concentration of analyte influences diffusion of the labeled analyte, as binding to antibodies slows diffusion of the labeled analyte through hydrogel 10. Analyte concentration can then be determined by measurement of the intensity of the labeled analyte across hydrogel 10.

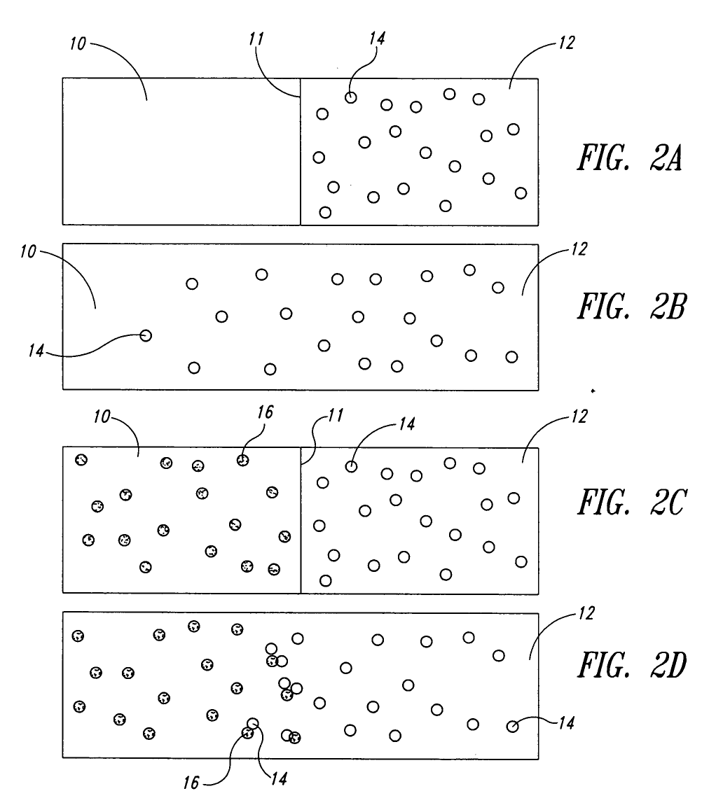

Referring now to FIG. 2A, there is shown the DIA of FIG. 1 after loading. Hydrogel 10 is placed in contact with sample 12, such as an aqueous solution like phosphate buffered saline, which contains analyte molecules 14. Hydrogel 10 contains no binding molecules. FIG. 2B shows the DIA of FIG. 2A after some time has elapsed. Analyte molecules 14 diffuse freely from sample 12 into hydrogel 10.

FIG. 2C shows hydrogel 10 in which binding molecules 16 such as an antibody or protein (albumin) are captured within hydrogel 10, and sample 12 contains analyte molecules 14 dispersed within the aqueous solution. In FIG. 2D, the DIA is shown after some time has elapsed. Binding molecules 16 remain trapped within hydrogel 10, while analyte molecules 14 are able to diffuse freely from sample 12 into hydrogel 10, where they bind with molecules 16, which causes an accumulation near the hydrogel—sample interface. This accumulation forms the diffusion front for this example. Analyte concentration can be measured by detecting the distribution of labeled analyte particles competing for antibody binding sites.

The contact area 11 between hydrogel 10 and sample 12 for this example was approximately 500 μm by 500 μm, or about 2500 μm². This interface 11 between hydrogel 10 and sample 12 may be of any shape or size which will not interfere with detection. For example, the interface may be rectangular as shown in FIG. 1, or hydrogels of any shape may be produced, such as in wells, and the carrier fluid for the analyte particles may be dropped into the hydrogels, or flowed over the hydrogels. Experiments show that the required device area was reduced by a factor of 50 when compared to flow conditions.

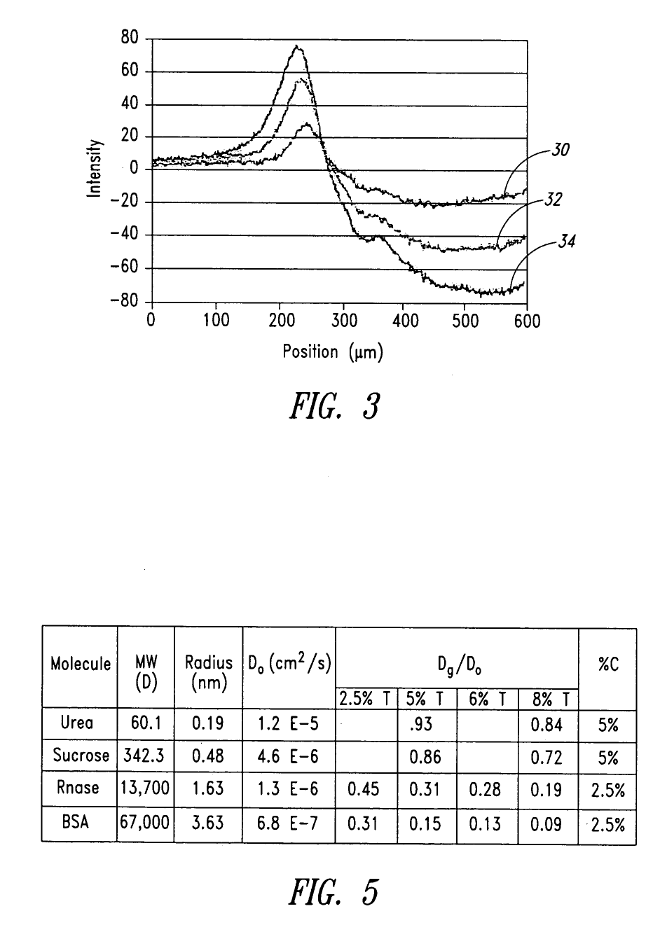

In one embodiment, biotin-specific antibody was immobilized in acrylamide hydrogels during polymerization. Hydrogels were generated with either photoinitiated polymerization or lithography techniques, or by standard reaction chemistry initiated by ammonium persulfates. Gels were cast between two glass coverslips separated by a 100 mm spacer layer. A 7.5% gel concentration with a 37.5:1 ratio of amylamide monomer: crosslinker was used. Fluorescein-biotin concentrations were 20 nM and biotin specific antibody concentrations were approximately 500 nM. The analyte accumulated at the edge of the hydrogel containing antibody. The results of three separate experiments are shown in FIG. 3, with the intensity shown as a function of position where the image data was processed at three time intervals: 60 seconds (shown at 30); 150 seconds (shown at 32); and 300 seconds (shown at 34).

Hydrogel devices for this invention have been designed to image binding reactions on a microscope system. For this design, the axis of optical interrogation is perpendicular to the device plane, and diffusion is observed across the d-dimension, as can be seen in FIG. 4. Acrylamide was chosen because it appeared to have the desired permeability to molecules of interest, it has been extensively studied, and it has proven to be compatible with biological samples including blood. Referring now to FIG. 4, there is seen a DIA device, designated at 20, having a hydrogel 22 in contact with a carrier sample 24. A detector device 26 is located above device 20, which observes diffusion across the d-dimension, interrogating volume 28 of device 20. Detector device 26 may include processing means, as earlier discussed, which can determine the presence or concentration of analyte particles within the diffusion front formed. This can be done by comparing the diffusion front with information stored in the processing means.

Two parameters are commonly adjusted to achieve the desired properties of an acrylamide gel; the percent acrylamide (reported as % T, total weight of acrylamide (monomer+cross-linker) per volume) and the percent crosslinker (reported as % C, weight of crosslinker per total weight of acrylamide). The acrylamide volume percent is also frequently measured after fabrication to account for the effects of hydrogel swelling, but swelling of native acrylamide has not been an issue for gels confined in microdevices and capillaries and no measurable swelling has been observed in initial experiments; % T will be the measure of acrylamide concentration in this work. For gel electrophoresis, % C is usually chosen based on the type of molecule being separated, with 5% C common for DNA and 2.6% C for protein separation. For a given % C, % T is usually adjusted to target a specific size range of protein or DNA; generally ranging from 5% to 15%. This gives some insight for designing microgels for diffusion analysis, but the conditions for electrophoresis are quite different since proteins are usually denatured for acrylamide electrophoresis, dramatically changing their shape (from globular to rod-like), and an electromotive force is applied.

Experimental measurements of molecular diffusion in acrylamide gels are more insightful for this application. The relevant data is summarized in the chart shown in FIG. 5, which shows the apparent diffusivity (D_{g}/Dₒ) of molecules in different gel concentrations, where Dₒ is the reported diffusion coefficient in saline at 20° C. For this application, the ideal hydrogel will negligibly affect the diffusion of small molecules while maximally reducing diffusion of the complex. For the acrylamide hydrogel proposed, ribonuclease is a protein slightly above a chosen small molecule cutoff (<10 kD) and bovine serum albumin (BSA) is a protein slightly above a chosen large molecule cutoff (>40 kD) giving a good indication of the limiting case for experiments with the proposed acrylamide gel system. Values of hydrogel permeability reported for these molecules indicate that a gel system with 2.6% C and % T up to 8% should be effective. Increasing acrylamide from 0 (free in solution) to 5% T resulted in a much greater diffusion differential between Rnase and BSA, with an acceptable reduction in the diffusivity of Rnase. Increasing acrylamide concentration from 0 to 8%, resulted in a change in their relative permeabilities from 2 to 30 indicating the increase in differential transport that can be obtained for the limiting case.

The efficiency of polymerization within microdevices is another important consideration. Photopolymerization has been tested with a range of % T from 2% to 15%. Gels photopolymerized with % T between 2-4% have been slower to form and have generally not filled the entire area patterned.

Although the examples shown in the application use acrylamide as the hydrogel, a number of hydrogels may be used, which vary in properties including pore structure, charge, responsiveness to environment, etc. Some of these hydrogels include agarose, poly(acrylamide), dextran, poly (vinyl alcohol), poly (ethylene oxide), poly (hydroxylethyl methacrylide), hydroxypropylmethyl cellulose, calcium alginate, and poly (ethylene glycol).

Examples of binding particles include antibodies, proteins, DNA, functionalized beads or even molecular imprinted beads.

Examples of analyte particles include therapeutic drugs, proteins, or any of a variety of molecules that would bind to a protein or antibody, or DNA.

Examples of labeled analytes include the above but conjugated to a fluorophore, chromophore, radiolabel, or some other measurable signal molecule.

While this invention has been shown and described in terms of preferred embodiments, it will be understood that this invention is not limited to any particular embodiment and that changes and modifications may be made without deporting from the true spirit and scope of the invention as defined in the appended claims.

What is claimed is:

1. A method for detecting the presence of analyte particles contained in a carrier allowing diffusion of said analyte particles, and method comprising the steps of:
a.) providing binding particles capable of binding with analyte particles;
b.) substantially immobilizing said binding particles in a hydrogel which permits diffusion of analyte particles;
c.) placing a carrier containing analyte particles in contact with said hydrogel;
d.) allowing said analyte particles to diffuse from said carrier into said hydrogel and bind with said binding particles;
e.) providing a detection device for detecting said binding particles or said analyte particles, or complexes thereof, or a diffusion front created by any of said particles;
and f.) detecting the position of any of said particles or diffusion front as an indication of the presence of said analyte particles.

2. The method of claim 1, wherein said diffusion front is detected at a predetermined time after initiating diffusion within the system.

3. The method of claim 1, wherein said binding particles are antibodies, proteins, DNA, or enzymes.

4. The method of claim 1, wherein said hydrogel is acrylamide.

5. The method of claim 1, wherein said analyte particles are supplemented with labeled analyte particles.

6. The method of claim 4, wherein said hydrogel comprises a solid.

7. The method of claim 1, wherein said detecting step comprises comparisons of the position of the diffusion front with the position of a diffusion front in a calibration system.

8. The method of claim 1, wherein said binding particles are labeled.

9. The method of claim 1, wherein said detection device is a CCD camera.

10. A microscale device for determining the presence or concentration of sample analyte particles, said device comprising:
a first section containing a carrier fluid containing analyte particles,
a second section containing a hydrogel in contact with said carrier fluid containing binding particles immobilized within said hydrogel which are capable of binding with analyte particles;
and a detection device for detecting a diffusion front formed by said analyte particles which have diffused into said hydrogel.

11. The device of claim 10, wherein said hydrogel is acrylamide.

12. The device of claim 10, wherein said detection device comprises a CCD camera.

13. The device of claim 10, further comprising means, coupled to said detection device, for determining from said detected diffusion front the presence or concentration of analyte particles.

14. The device of claim 10, wherein the area of contact between said hydrogel and said carrier fluid comprises approximately 2500 μm².

15. The device of claim 10, wherein the area of contact between said hydrogel and said carrier fluid is rectangular.
